(19) United States
(12) Patent Application Publication
(10) Pub. No.: US 2021/0310950 A1
(43) Pub. Date: Oct. 7, 2021

(54) DEVICE, PROCESS AND SYSTEM FOR GEMOLOGICAL CHARACTERIZATION

(71) Applicant: GOLDWAY TECHNOLOGY LIMITED, Hong Kong (CN)

(72) Inventors: Juan CHENG, Hong Kong (CN); Chun Hong SHAM, Hong Kong (CN); Tat Hang CHUNG, Hong Kong (CN); Kong CHAN, Hong Kong (CN); Wing Chi TANG, Hong Kong (CN); Koon Chung HUI, Hong Kong (CN)

(73) Assignee: GOLDWAY TECHNOLOGY LIMITED, Hong Kong (CN)

(21) Appl. No.: 17/265,088

(22) PCT Filed: Aug. 1, 2019
(86) PCT No.: PCT/CN2019/098937

§ 371 Date: Feb. 1, 2021

(30) Foreign Application Priority Data
Aug. 1, 2018 (CN) 201821229747.4
Aug. 1, 2018 (CN) 201821230913.2
Aug. 1, 2018 (CN) 201821231063.8

(51) Int. Cl. G01N 21/64; G01N 21/87

(52) U.S. Cl. CPC G01N 21/645; G01N 21/65; G01N 2021/6471; G01N 21/87

(57) ABSTRACT

A system (100a) for determining the type of a diamond (130a), the system (100a) comprising a plurality of lasers (110a,120a) for directing light towards a diamond (130a), wherein each laser is of a different wavelength of light; wherein the spectrum of light from the plurality of lasers (110a,120a) extends from ultra-violet to near infra-red; a spectrometer (140a) for collecting luminescence spectrum from the diamond (130a) responsive to inhomogeneities upon light from the lasers (110a,120a) being directed towards the diamond (130a); a processor module (150a) for comparing photoluminescence spectrum collected by the spectrometer (140a) with pre-existing photoluminescence spectrum of known diamond type; and an output module (160a) for providing an output signal indicative of the diamond type of the diamond (130a), upon a predetermined threshold of correlation between the photoluminescence spectrum from the diamond (130a) and the pre-existing photoluminescence spectrum from the diamond (130a) and the pre-existing photoluminescence spectrum responsive to inhomogeneities of known diamond type.

### TECHNICAL FIELD

The present invention relates to a device, process and system for gemological characterization. More particularly, the present invention provides a device, process and system for characterizing a diamond.

### BACKGROUND OF THE INVENTION

A natural diamond is considered a rare gemstone, and natural diamonds have been considered to be formed often between 1 billion and 3.5 billion years ago, with most formed at depths between 150 and 250 kilometres below the surface of the earth.

Parameters of a diamond, the clarity, cut, carat and colour of a diamond influence the value of a diamond.

As is known, diamonds of higher value are typically those of very little or no discernable colour, which is typically a subtle yellow tinge.

Further, diamonds of higher clarity, that is with fewer visible defects or inclusions with the body of the diamond, are of higher economic value.

As in more recent years, synthetic or non-natural diamonds have been produced, which a formed or grown in a laboratory, and man-made, which are made in a controlled laboratory environment that purported to reflect the conditions needed for diamonds to form in nature.

There are two main processes to create man-made diamonds; chemical vapor deposition (CVD diamonds) and high-pressure high treatment (HPHT diamonds);
(i) A CVD (chemical vapor deposition) diamond is a laboratory made diamond, which is created through the process of chemical vapor deposition. This method is often used for large stones.
(ii) an HPHT (high pressure high temperature) diamond is a laboratory made diamond used with a process called high pressure high treatment. HPHT is primarily used for small diamond melee, not usually for larger stones.

Laboratory made diamonds are still considered to be real diamonds, and are comprised of mineral consisting of pure carbon crystallized in the isometric system, and the differences are indistinguishable to the naked eye and nearly if not impossible under magnification.

Synthetically formed diamonds are considered to be “real”, and grading authorities may issue one report for natural diamonds and a separate report for laboratory made diamonds.

Non-natural (i.e laboratory made) diamonds are generally considered to be of a lower economic value and can be considered non-authentic or at least non-traditional.

As part of the value of a natural diamonds, the age, millions or billions of years, and the scarcity and unique nature between every diamond, drives the value of such diamonds.

Further, the history of a diamond also may contribute to its value, and at least sentimental value when a diamond has been gifted or passed down through generations in a family.

The advent of high quality synthetically formed diamonds, such as CVP and HPHT diamonds, has had a significant effect in the diamond industry.

As is known, there have been numerous instances of natural diamonds being replaced with synthetic diamonds, as part of fraudulent activities, with the real owner not being aware of such deceit.

Also, there have been numerous instances of high-quality synthetic diamonds being passed off to customers as being real diamonds, or real diamonds having full documentation being substituted by synthetic diamonds between purchase and collection.

However, due to the great advance in CVD and HPHT technologies for synthetic diamonds in recent years, making discernment increasingly difficult between the different types.

Furthermore, some low-grade natural diamonds can even be treated with HPHT to become high grade diamonds, thus modifying the value of a diamond whilst representing the diamond to be naturally occurring at that grade.

At present, Raman spectrometers can be used for diamond type determination, that is generally natural or synthetic, and these are mostly portable Raman spectrometers.

With the increasing similarity of synthetic diamonds to natural diamonds, and the increasing difficulty to determine the type of diamonds, that is natural and unmodified diamonds, versus synthetic or modified natural diamonds, has become increasingly difficult, and existing processes for determining diamond type are increasingly less reliable and uncertain and inevitably shall become obsolete in the near future. Therefore, new methodologies to identify natural, synthetic and treated diamonds are needed.

### OBJECT OF THE INVENTION

It is an object of the present invention to provide a device, process and system for gemological characterization, in particular a diamond and determining the type of diamond, which overcomes or at least partly ameliorates at least some deficiencies as associated with the prior art.

### SUMMARY OF THE INVENTION

In a first aspect, the present invention provides system for determining the type of a diamond, said system comprising:
a plurality of lasers for directing light towards a diamond, wherein each laser is of a different wavelength of light; wherein the spectrum of light from said plurality of lasers extends from ultra-violet (UV) to near infra-red (NIR);
a spectrometer for collecting photoluminescence spectrum from said diamond responsive to inhomogeneities upon light from said lasers being directed towards said diamond;
a processor module for comparing photoluminescence spectrum collected by the spectrometer with pre-existing photoluminescence spectrum of known diamond type; and
an output module for providing an output signal indicative of the diamond type of said diamond, upon a predetermined threshold of correlation between the photoluminescence spectrum from said diamond and said pre-existing photoluminescence spectrum responsive to inhomogeneities of known diamond type.

The spectrometer may collect photoluminescence spectrum intensity data from said diamond whilst all lasers are simultaneously activated.

The inhomogeneities include colour centers, inclusion, defects, crystallinity inconsistency, deformation of crystal lattice, internal stress, internal stress, impurities and trace elements.

The types of diamond include natural diamonds chemical vapor deposition (CVD) synthetic diamonds, high pressure high temperature (HPHT) synthetic diamonds, and treated natural diamonds.

The system may comprise 3 lasers.

The system may comprise 4 lasers. The lasers preferably have wavelengths of 360 nm, 457 nm, 514 nm and 633 nm.

In a second aspect, the preset invention provides a process for determining the type of a diamond, said process including the steps of:

(i) collecting photoluminescence spectrum from said diamond responsive to inhomogeneities upon light from a plurality of lasers, wherein each laser is of a different wavelength of light; wherein the spectrum of light from said plurality of lasers extends from ultra-violet (UV) to near infra-red (NIR);

(ii) in a processor module, comparing the photoluminescence spectrum collected by the spectrometer with pre-existing photoluminescence spectrum of known diamond type; and

(iii) from an output module, responsive to predetermined threshold of correlation between the photoluminescence spectrum from said diamond and said pre-existing photoluminescence spectrum of known diamond type from step (ii) an output signal is provided indicative of the type of the diamond.

The spectrometer may collect photoluminescence spectrum intensity data from said diamond whilst all lasers are simultaneously activated.

The inhomogeneities include colour centers, inclusion, defects, crystallinity inconsistency, deformation of crystal lattice, internal stress, internal stress, impurities, trace elements and isotopes.

The types of diamond include natural diamonds chemical vapor deposition (CVD) synthetic diamonds, high pressure high temperature (HPHT) synthetic diamonds, and treated natural diamonds.

The pre-existing photoluminescence spectrum intensity data of known diamond type may be acquired using the system of the first aspect.

In a third aspect, the present invention provides a system for automatically optimizing a collection spectrum in diamond detection, said system comprising a belt worktable, wherein a microscope objective lens and an optical device are respectively arranged on both sides of the belt worktable, a laser source is arranged in front of the microscope objective lens, a CCD sensor is arranged behind the optical device, the CCD sensor performs pre-acquisition, and the CCD sensor adjusts its own parameters according to a pre-acquisition result.

The microscope objective lens may be fixed to a support frame.

The laser source may be fixed to a mounting table.

The laser source may comprise different lasers.

The CCD sensor may be connected to a spectrometer.

The CCD sensor preferably adopts an area array CCD.

The optical device may be a notch filter and a fluorescence filter.

In a fourth aspect, the present invention provides a diamond detection device for simultaneous co-point excitation with multiple laser lights comprising a bottom plate and a worktable, wherein the top of the bottom plate and the bottom of the worktable are both oppositely fixed with connecting rods, the connecting rods are connected by springs, the top of the bottom plate and the bottom of the worktable are both fixed with seats between the connecting rods, an air bag cooperating with the seats is arranged between the seats, a damping ring is arranged between the connecting rod and the seat, the top of the bottom plate and the bottom of the worktable are both oppositely fixed with support plates, second spring rods are oppositely fixedly connected between the support plates, elastic balls are arranged between the second spring rods, the end of the support plate is fixed with an elastic plate, a belt worktable is arranged above the worktable, a microscope objective lens and an optical device are oppositely arranged on both sides of the belt worktable, a dichroic mirror is arranged in front of the microscope objective lens, a fluorescence filter is arranged behind the optical device, the dichroic mirror, the microscope objective lens, the belt worktable, the optical device and the fluorescence filter are all fixed to the worktable through supporting rods, the supporting rod located at the bottom of the dichroic mirror is fixed to one end of a connecting rod, the other end of the connecting rod is fixed with a mounting plate, and the mounting plate is fixed with a laser source.

The seats may be symmetrically arranged in a vertical direction.

The support plates may be symmetrically arranged in a vertical direction.

The cross section of the support plate may be “L” shaped.

The second spring rods may be symmetrically arranged in a transverse direction.

The connecting rod is preferably “L” shaped.

The laser source may comprise different lasers.

The dichroic mirror preferably reflects a laser wavelength, and the dichroic mirror preferably transmits a fluorescence wavelength.

The optical device is preferably a laser line filter and a spatial filter.

In a fifth aspect, the present invention multi-purpose optical detection system with fiber coupling comprising a bottom plate and a worktable, wherein the bottom plate and the worktable are fixedly connected by first spring rods, the side surface of the first spring rod is oppositely connected with transverse spring rods, springs are arranged both between the transverse spring rod and the bottom plate and between the transverse spring rod and the worktable, a damping cylinder is arranged outside the transverse spring rod and the springs, mounting plates and damping pads are sequentially fixed at intervals outside the damping cylinder, the top of the bottom plate and the bottom of the worktable are oppositely fixed with support plates, second spring rods are oppositely fixedly connected between the support plates, elastic balls are arranged between the second spring rods, the end of the support plate is fixed with an elastic plate, a laser source, a first fiber coupler, a first optical device, a placement table, a second optical device and a second fiber coupler are sequentially fixed from left to right on the worktable, the laser source is connected with the first fiber coupler through fibers, the second fiber coupler is connected with different spectrometers through fibers, side plates are oppositely fixed on the placement table, and the side plates are connected with fixed blocks through the springs.

The first spring rods are preferably symmetrically arranged in a transverse direction.

The support plates may be symmetrically arranged in a vertical direction.

The cross section of the support plate may be “L” shaped.

The second spring rods may be symmetrically arranged in a transverse direction.

The laser source may comprise different lasers.

The first optical device is preferably a lens, a dichroic mirror and a microscope objective lens, and the second optical device is a notch filter and a fluorescence filter.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1a shows a schematic diagram of the structure of a gemological characterization system according to the present invention;

FIG. 1b shows a further schematic diagram of the structure of an embodiment of a gemological characterization system according to the present invention;

FIG. 2 shows a schematic diagram of the structure of an automatically optimizing spectra collection device according to the present invention;

FIG. 3 shows a schematic diagram of the structure of a gemological characterization device according to the present invention having multi-laser simultaneous excitation at same position and spectra simultaneous collection, whereby multiple lasers are simultaneously excited and collected at the same point;

FIG. 4a shows a schematic diagram of the structure of an embodiment of the gemological characterization system according to FIG. 3;

FIG. 4b shows a schematic diagram of the top view structure of the placement table of the gemological characterization system of FIG. 4a;

FIG. 5a shows a photoluminescence spectrum from a natural diamond when it is simultaneously excited by four lasers with different wavelength;

FIG. 5b shows a photoluminescence spectrum from a CVD diamond when it is simultaneously excited by four lasers with different wavelength;

FIG. 5c shows a photoluminescence spectrum from a HPHT diamond when it is simultaneously excited by four lasers with different wavelength; and

FIG. 6 shows a photoluminescence spectrum from a HPHT diamond when it is simultaneously excited by four lasers with wavelengths different to that in FIGS. 5a-5c.

### DETAILED DESCRIPTION OF THE DRAWINGS

### 1. Prior Art and Identified Deficiencies

At present, Raman spectrometers used for diamond detection on the market are mostly portable Raman spectrometers, which use single-wavelength laser lights to excite samples. The use of light and portable spectrometers to acquire signals brings certain limitations to the spectrum range of the detection signal and the resolution of the spectrum. Different impurity components in the diamond have different excitation responses to laser lights of different wavelengths.

When a Raman spectrum is traditionally used to assist in the analysis and detection of a diamond sample, the power of the laser light incident on the surface of the sample, the size of the slit of the spectrometer, and the response parameters of the CCD should be correspondingly manually adjusted according to different samples, which will make the entire detection flow take a lot of time, resulting in a reduced detection efficiency.

Some jewellery and jade identification agencies adopt laser confocal micro-Raman spectrometers with high sensitivities and high spatial resolutions to identify and detect diamonds. Although the spectrometer can perform an excitation test on a sample with multiple laser lights of different wavelengths, since a single spectrometer acquires signal lights, as for the same sample, it generally uses laser lights of different wavelengths to perform excitation one by one, and then collects signal lights one by one for subsequent analysis.

In addition, the sample compartment of the spectrometer is comparatively small, and the travel of the electric displacement table thereof is limited, thereby limiting the number of samples placed in the sample tray each time, which greatly slows down the detection speed of the diamonds, and is far from meeting the demand for batch, fast and accurate detection of diamond samples on the market.

In addition, the structure of the existing detection device is not sufficiently stable, and the vibration of the device will affect the accuracy of the detection result.

Further, conventional optical detection systems (Raman spectroscopy, photoluminescence (PL in short) spectroscopy) all use laser lights of specific wavelengths to excite samples, and use fixed spectrometers to acquire signal lights.

When it is required to perform optical detection of samples at different positions or of different properties, it is generally required to move the laser or move the spectrometer to rebuild the optical system to perform the detection.

This will make the entire detection flow time-consuming and laborious, and especially when the spectrometer is cumbersome or the laser cannot move, this will make the detection more difficult.

As will be understood, in addition to being cumbersome, the prior art is exposed to errors such as between readings with different lasers excitations, diamonds may be moved or changes, giving rise to the potential of inconsistent readings, which can result in mis-classification of a diamond type, which introduces the propensity for non-detection of synthetic diamonds, as well as the propensity for a synthetic diamond to be considered as a natural diamond.

### 2. Use of Photoluminescence (PL) in Diamond Type Determination

The laser excited photoluminescence (PL) spectrum has become an essential tool used to separate treated and synthetic diamonds from their natural counterparts.

Atomic-scale features (often termed as optical centers or colour centers) occur within the diamond structure, examples include carbon, nitrogen, boron, and vacancies in the lattice such as nitrogen-vacancy, silicon vacancy, boron-vacancy for example.

The configuration of these defects varies with the growth conditions and subsequent geological or treatment history. PL can provide a very sensitive tool for detecting deviations in atomic configurations and defects even at concentrations of less than ten in a billion carbon atoms.

For example, the N3 center (415 nm peak) is a very common optical feature in most type Ia natural diamonds containing the B-aggregates of nitrogen. The PL peak at 737 nm (the SiV⁻) indicates the presence of silicon impurities in diamond, which is very rarely seen in natural diamond but typically occur in CVD synthetic diamond and thus assist in their identification.

While, the 882 nm peak is a center observed in nickel- and nitrogen-containing synthetic diamonds grown by the temperature gradient method used in high pressure high temperature (HPHT) synthetic process.

For these feature peaks, most of them are excitation-dependent emission, i.e. some peak cannot be excited by a short wavelength laser but can be excited by a longer wavelength laser, which make it necessary to have a full range PL spectra from UV to NIR excited with different wavelength lasers.

### 3. Present Invention

The present invention provides a process and a system, which reliably, expediently and consistently determines the type of diamond being analysed.

Such a process and system of the present invention has:
(1) the full range photoluminescence spectra are collected simultaneously and excited by multi-laser at same time, which can greatly improve test efficiency.
(2) an excitation area on the sample is similar by the multi-laser, thus providing more spectra information on the same position of the sample.

Aspects and embodiments of the present invention further provide:
(i) a motorized three-dimensional (3D) stage having a long travel distance, allowing for the analysis of several hundred samples in a fixture at one time.
(ii) an acquisition system with automatic optimization and high-resolution spectrometer to collect signal, we can obtain high SNR (signal to noise ratio) and high resolution as well as high reliability data results. This provides for comparison of the results with the existing database, such so that a samples' properties may be assessed
(iii) the using of a fiber coupled system in the excitation and collection part, which provides for ease of change the laser source and collection system when it is necessary. Also, the lasers and spectrograph can achieve free placement in space.

### 4. Advantages of the Present Invention and Embodiments Thereof

Advantages of the present invention include:
(a) reducing misclassification of a diamond type, and thus decreasing the propensity for non-detection of synthetic diamonds, as well as the propensity for a synthetic diamond to be considered as a natural diamond;
(b) a large photoluminescence spectral range spanning from UV to NIR with the right combination of excitation lasers and dichroic beam splitters, and thus allowing simultaneous assessment of different types of colour center;
(c) with a motorized three-dimension 3D stage, sample can reside anywhere within an area for example of 150 mm×150 mm, making it possible to perform automatic measurements;
(d) using the automated analysis platform, the full photoluminescence spectrum performance of the diamond samples can be quickly detected, and the detection speed can reach 1150 particles/hour;
(e) reduction in manpower, and reduction errors that may be caused by human actions; and
(f) increased reliability, certainty and reproducibility.

### 5. Definitions

For the purposes of this invention, inhomogeneities of a diamond are understood to include colour centers, inclusion, defects, crystallinity inconsistency, deformation of crystal lattice, internal stress, internal stress, impurities, trace elements and isotopes.

For the purposes of this invention, the types of diamond are understood to be natural diamonds chemical vapor deposition (CVD) synthetic diamonds, high pressure high temperature (HPHT) synthetic diamonds, and treated natural diamonds.

Such inhomogeneities, depending on the type and characteristics thereof, cause different intensity characteristics of photoluminescence upon illumination with light of different wavelengths.

### 6. Invention

The present invention provides a system and a process for determining the type of a diamond.

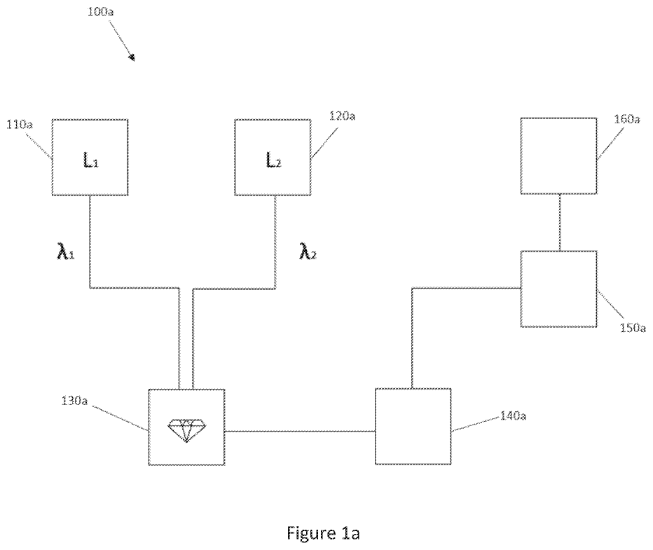

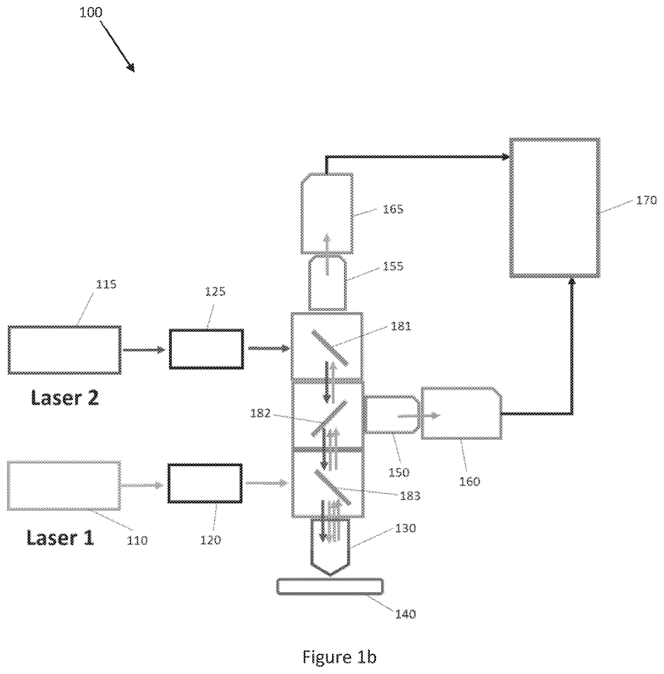

In accordance with the invention and referring to FIG. 1a, a system 100a in accordance with the present invention is schematically represented, and comprises a plurality of lasers 110a, 120a for directing light towards a diamond 130a, wherein each laser 110a, 120a and is of a different wavelength λ₁, λ₂, of light; wherein the spectrum of light from said plurality of lasers 110a, 120a, extends from ultra-violet (UV) to near infra-red (NIR);

The system 100a includes a spectrometer 140a for collecting photoluminescence spectrum intensity data from the diamond 130a responsive to inhomogeneities upon light from said lasers 110a, 120a, being directed towards said diamond 130a.

A processor module 150a is provided for comparing photoluminescence spectrum intensity data collected by the spectrometer 140a with pre-existing photoluminescence spectrum intensity data of known diamond type.

The system further comprises an output module 160a in communication with the processor module 150a for providing an output signal indicative of the diamond type of the diamond 130a, upon a predetermined threshold of correlation between the photoluminescence spectrum intensity data from the diamond 130a and pre-existing photoluminescence spectrum intensity data responsive to inhomogeneities of known diamond type.

Referring to FIG. 1b, there is shown a further embodiment of a system 100 of the present invention which also embodies the process of the present invention.

The system has a single mode fiber coupled laser 1 and laser 2, 110 and 115, that are collimated by fiber collimators, 120, 125, then reflected to the objective lens 130.

The two collimated lasers are focused simultaneously on the sample surface 140 via the objective lens 130 at a same point. The photoluminescence excited by the two lasers 110, 115, are collected by the spectrometer 160, 165, via the collection lens systems of 150, 155. Then the signal data are recorded by a processor such as a PC 170.

In the setup of the system 100, items 181 and 183 are long pass dichroic beam splitters with different wavelengths range corresponding to the excitation laser wavelengths.

The long pass dichroic beam splitter 182 reflects spectrum wavelengths shorter than that of laser 115 and transmits wavelengths equal and longer than laser 115.

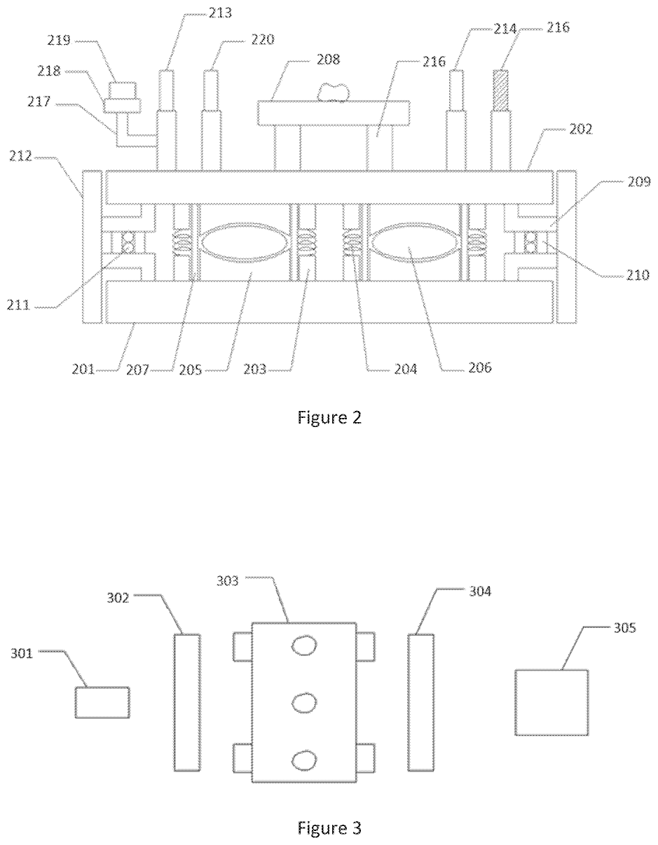

Referring to FIG. 2, there is shown a system for automatically optimizing a collection spectrum in diamond detection, which comprises a belt worktable 203, wherein a microscope objective lens 202 and an optical device 204 are respectively arranged on both sides of the belt worktable 203, a laser source 201 is arranged in front of the microscope objective lens 202, a CCD sensor 205 is arranged behind the optical device 204, the CCD sensor 205 performs pre-acquisition, and the CCD sensor 205 adjusts its own parameters according to a pre-acquisition result.

The microscope objective lens 202 is fixed to a support frame.

The laser source 201 is fixed to a mounting table.

The laser source 201 comprises different lasers.

The CCD sensor 205 is connected to a spectrometer.

The CCD sensor 205 adopts an area array CCD.

The optical device 204 is a notch filter and a fluorescence filter.

In use, the diamond sample to be detected is placed on the belt worktable 203, and the microscope objective lens 202 can focus the laser lights generated by the lasers of different wavelengths in the laser source 201 on the diamond sample. After filtration by the optical device 204, the CCD sensor 205 performs the pre-acquisition, the pre-acquisition does not require a high signal-to-noise ratio, so the speed is very fast, then the CCD sensor 205 adjusts its own parameters according to the pre-acquisition result, thereby obtaining a Raman spectrum having a comparatively high signal-to-noise ratio, and then the Raman spectrum of the sample is compared with those of the diamonds of different types to judge whether the sample belongs to the diamond and its classification

The actual numbers and designs of the laser sources 201, the samples, and the CCD sensors 205 can be set as required. The laser source 201, the device for placing the sample, and the CCD sensor 205 may have different designs, which may be composed of different components.

It should be noted that the optical device 204, which is composed of a notch filter and a fluorescence filter, serves the purpose of filtering out non-signal lights.

The system for automatically optimizing a collection spectrum in diamond detection provided by the present invention can utilize the microscope objective lens to focus the laser lights to excite the diamond samples at different positions on the belt worktable, after filtration by the optical device, the CCD sensor performs pre-acquisition, the pre-acquisition does not require a high signal-to-noise ratio, so the speed is very fast, then the CCD sensor adjusts its own parameters according to the pre-acquisition result, thereby obtaining a Raman spectrum having a comparatively high signal-to-noise ratio, and then the Raman spectrum of the sample is compared with those of the diamonds of different types to judge whether the sample belongs to the diamond and its classification, so that the flow of collecting the Raman spectrum can be automatically optimized to solve the problem that the detection flow takes a comparatively long time and reduce the time required to measure multiple samples, and meanwhile human errors can be also reduced.

Referring to FIG. 3, there is shown a diamond detection device for simultaneous co-point excitation with multiple laser lights, wherein the diamond detection device comprises a bottom plate 301 and a worktable 302, wherein the top of the bottom plate 301 and the bottom of the worktable 302 are both oppositely fixed with connecting rods 303, the connecting rods 303 are connected by springs 304, the top of the bottom plate 301 and the bottom of the worktable 302 are both fixed with seats 305 between the connecting rods 303, an air bag 306 cooperating with the seats 305 is arranged between the seats 305, a damping ring 307 is arranged between the connecting rod 303 and the seat 305, the top of the bottom plate 301 and the bottom of the worktable 302 are both oppositely fixed with support plates 309, second spring rods 310 are oppositely fixedly connected between the support plates 309, elastic balls 311 are arranged between the second spring rods 310, the end of the support plate 309 is fixed with an elastic plate 312, a belt worktable 308 is arranged above the worktable 302, a microscope objective lens 320 and an optical device 314 are oppositely arranged on both sides of the belt worktable 308, a dichroic mirror 313 is arranged in front of the microscope objective lens 320, a fluorescence filter 315 is arranged behind the optical device 314, the dichroic mirror 313, the microscope objective lens 320, the belt worktable 308, the optical device 314 and the fluorescence filter 315 are all fixed to the worktable 302 through supporting rods 316, the supporting rod 316 located at the bottom of the dichroic mirror 313 is fixed to one end of a connecting rod 317, the other end of the connecting rod 317 is fixed with a mounting plate 318, and the mounting plate 318 is fixed with a laser source 319.

The seats 305 are symmetrically arranged in a vertical direction, the support plates 309 are symmetrically arranged in a vertical direction, the cross section of the support plate 309 is “L” shaped, the second spring rods 310 are symmetrically arranged in a transverse direction, the connecting rod 317 is “L” shaped, the laser source 319 comprises different lasers, the dichroic mirror 313 reflects a laser wavelength, the dichroic mirror 313 transmits a fluorescence wavelength, and the optical device 314 is a laser line filter and a spatial filter.

In use, the diamond sample to be detected is placed on the belt worktable 308, the laser source 319 is debugged to a state suitable for detection, the dichroic mirror 313 reflects the laser wavelength and transmits the fluorescence wavelength, the microscope objective lens 320 performs focusing to excite the sample, the optical device 314 filters out non-signal lights, the fluorescence filter 315 distinguishes signal lights in different fluorescence bands, a plurality of spectrometers with high resolutions are used to simultaneously acquire Raman/photoluminescence spectrums in different wave bands generated by laser lights of different wavelengths, and the spectrum of the diamond sample is compared with those of the diamonds of different types to achieve the identification of the sample.

On the premise that the optical component and the fluorescence filter 315 are selected correctly, the system may have two or more laser sources 319 and may also have two or more spectrometers, and the number of the laser sources 319 is not necessarily the same as the number of the spectrometers. The laser source 319, the spectrometer, the device for placing the sample, and the device for distinguishing signal lights in different wave bands may have their actual designs performed as required, which may be composed of different components.

The springs 304 between the connecting rods 303 in cooperation with the air bags 306 between the seats 305 can effectively slow down the vibration between the worktable 302 and the bottom plate 301, the second spring rods 310 and the elastic balls 311 between the support plates 309 can also slow down the vibration between the worktable 302 and the bottom plate 301, so that the overall structure is more stable, and the impact on the accuracy of the detection result brought by the vibration is effectively reduced.

The diamond detection device for simultaneous co-point excitation with multiple laser lights provided by the present invention can adopt a plurality of lasers of different wavelengths to perform simultaneous co-point excitation on the same sample, use the dichroic mirror to reflect the laser wavelength and transmit the fluorescence wavelength, use the microscope objective lens to perform focusing to excite the sample, use the optical device to filter out non-signal lights, use the fluorescence filter to distinguish signal lights in different fluorescence bands, and use a plurality of spectrometers with high resolutions to simultaneously acquire Raman/photoluminescence spectrums in different wave bands generated by laser lights of different wavelengths, and thus can enlarge the range of the detection spectrum of the spectrometer, improve the resolution of the spectrum, and improve the detection speed of the diamond samples.

The springs between the connecting rods in cooperation with the air bags between the seats can effectively slow down the vibration between the worktable and the bottom plate, the second spring rods and the elastic balls between the support plates can also slow down the vibration between the worktable and the bottom plate, so that the overall structure is more stable, and the impact on the accuracy of the detection result brought by the vibration is effectively reduced.

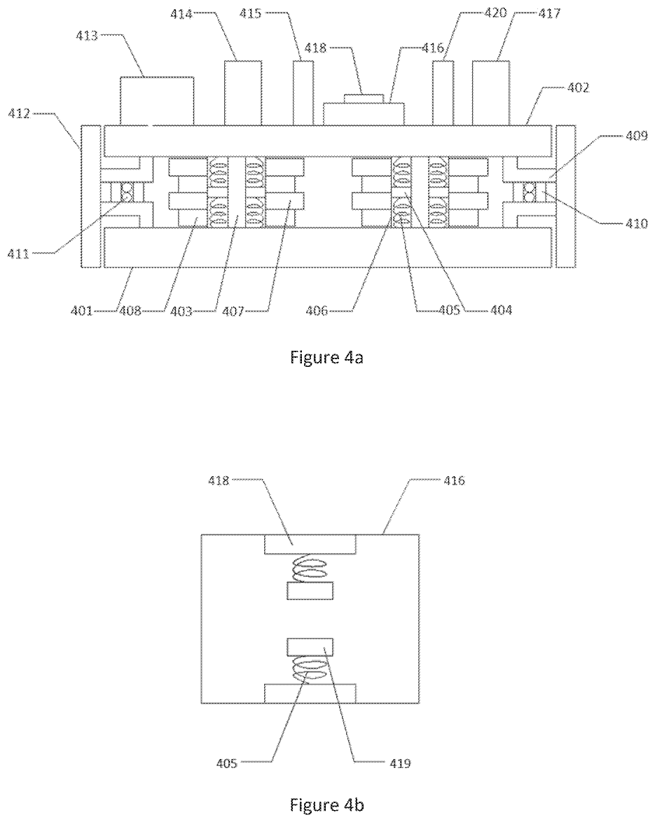

A multi-purpose optical detection system with fiber coupling, as shown in FIG. 4a and FIG. 4b, comprises a bottom plate 401 and a worktable 402, wherein the bottom plate 401 and the worktable 402 are fixedly connected by first spring rods 403, the side surface of the first spring rod 403 is oppositely connected with transverse spring rods 404, springs 405 are arranged both between the transverse spring rod 404 and the bottom plate 401 and between the transverse spring rod 404 and the worktable 402, a damping cylinder 406 is arranged outside the transverse spring rod 404 and the springs 405, mounting plates 407 and damping pads 408 are sequentially fixed at intervals outside the damping cylinder 406, the top of the bottom plate 401 and the bottom of the worktable 402 are oppositely fixed with support plates 409, second spring rods 410 are oppositely fixedly connected between the support plates 409, elastic balls 411 are arranged between the second spring rods 410, the end of the support plate 409 is fixed with an elastic plate 412, a laser source 413, a first fiber coupler 414, a first optical device 415, a placement table 416, a second optical device 420 and a second fiber coupler 417 are sequentially fixed from left to right on the worktable 402, the laser source 413 is connected with the first fiber coupler 414 through fibers, the second fiber coupler 417 is connected with different spectrometers through fibers, side plates 418 are oppositely fixed on the placement table 16, and the side plates 418 are connected with fixed blocks 19 through the springs 5.

The first spring rods 403 are symmetrically arranged in a transverse direction, the support plates 409 are symmetrically arranged in a vertical direction, the cross section of the support plate 409 is “L” shaped, the second spring rods 410 are symmetrically arranged in a transverse direction, the laser source 413 comprises different lasers, the first optical device 415 is a lens, a dichroic mirror and a microscope objective lens, and the second optical device 420 is a notch filter and a fluorescence filter.

In use, the sample to be detected is fixed on the placement table 416 using the springs 405 and the fixed blocks 419, and collimation debugging is performed by means of the first optical device 415 so that the laser lights coupled by the first fiber coupler 414 are focused on the surface of the sample.

The first fiber coupler 414 is capable of coupling lights emitted from different lasers in the laser source 413, the lights being collimated and focused on the sample after passing through the first optical device 415, and then entering the second fiber coupler 417 by passing through the second optical device 420 to thereby enter different spectrometers, and the Raman spectrum of the sample is compared with the spectrums of the diamonds of different types to judge whether the sample belongs to the diamond and its classification.

The user can reconnect the first fiber coupler 414 to a different laser source 413 and reconnect the second fiber coupler 417 to a different sensor as needed for the next measurement. The actual numbers of the laser sources 413 and the spectrometers can be set according to different requirements, the actual designs of the laser source 413, the spectrometer and the device for placing the sample can be set according to different requirements, different optical components may be arranged in the device for placing the sample, and connecting fibers may also exist therein for switch to different components.

The first spring rods 403 in cooperation with the springs, the mounting plates 407 and the damping pads 408 can effectively slow down the vibration between the worktable 402 and the bottom plate 401, the second spring rods 410 and the elastic balls 411 between the support plates 409 can also slow down the vibration between the worktable 402 and the bottom plate 401, so that the overall structure is more stable, and the impact on the accuracy of the detection result brought by the vibration is effectively reduced.

It should be noted that the first optical device 415, which is composed of a lens, a dichroic mirror and a microscope objective lens, serves the purpose of performing collimation debugging of the optical system and focusing the laser lights on the surface of the sample, and the microscope objective lens is also used for collecting signal lights. The second optical device 420, which is composed of a notch filter and a fluorescence filter, serves the purpose of filtering out non-signal lights

The multi-purpose optical detection system with fiber coupling provided by the present invention adopts a fiber coupling manner to connect fibers that can be easily removed and installed to different laser sources, then performs collimation debugging of the optical system by means of the first optical device, and focuses the laser lights on the surface of the sample, in the part of the light-receiving system, non-signal lights can be filtered out by means of the second optical device, and the fiber coupling manner is also adopted to collect signal lights using fibers and couple the lights into the spectrometer, so that the user can transfer excitation lights from the fibers to an optical path where the sample is placed to detect the sample according to different requirements, and then the signal lights are transmitted into the spectrometer by the fibers, which may reduce the time and manpower for switching the laser source and the spectrometer by the detection system, and meanwhile reduce human errors. The first spring rods in cooperation with the springs, the mounting plates and the damping pads can effectively slow down the vibration between the worktable and the bottom plate, and the second spring rods and the elastic balls between the support plates can also slow down the vibration between the worktable and the bottom plate, so that the overall structure is more stable, and the impact on the accuracy of the detection result brought by the vibration is effectively reduced.

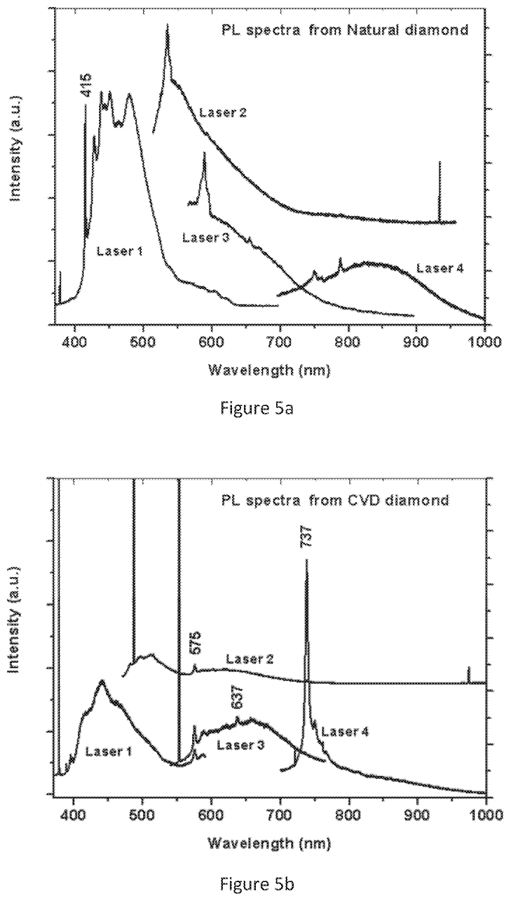

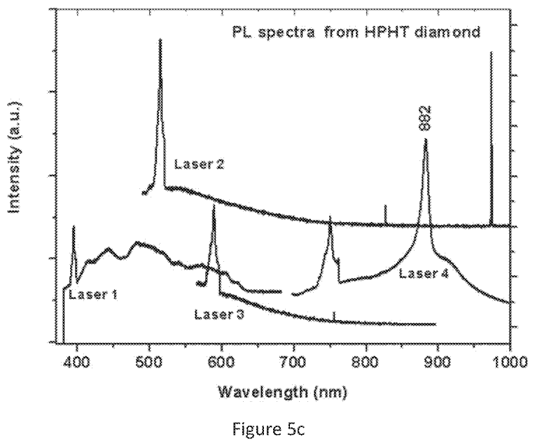

FIG. 5a-5c are the photoluminescence spectra obtained from diamonds when it is simultaneously excited by four lasers with different wavelengths. The four lasers are:

Laser 1—160 nm

Laser 2—457 nm

Laser 3—514 nm

Laser 4—633 nm

FIG. 5a shows the photoluminescence spectra for Natural diamonds for these four above wavelengths, FIG. 5b shows the photoluminescence spectra for CVD diamonds for these four wavelengths, and FIG. 5c shows the photoluminescence spectra for HPTP diamonds for these four wavelengths. As can be seen there are different characteristic peaks when they are excited by the same lasers, and such data is utilized in determining the type of diamond in accordance with the present invention.

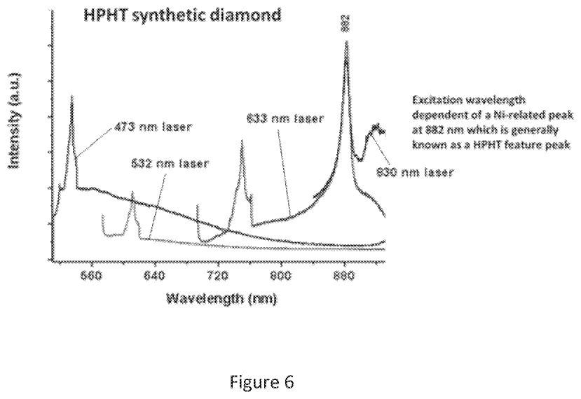

Referring to FIG. 6, the wavelength of each individual laser can be adjusted, in order to obtain the most optimal results in some cases, and as is shown the photoluminescence spectra for a HPHT diamond with different wavelengths as utilized for the HPHT diamond in FIG. 5c.

The above embodiment is only used to describe the technical solution of the present invention, and not to limit it. Although the present invention is described in detail with reference to the above-mentioned embodiment, those skilled in the art should understand that they can still make amendments to the technical solution recorded in the above-mentioned embodiment or make equivalent substitutions of parts of the technical features therein, and these amendments or substitutions will not make the essence of the corresponding technical solution break away from the spirit and scope of the embodiment of the present invention.

1. A system for determining the type of a diamond, said system comprising:
a plurality of lasers for directing light towards a diamond, wherein each laser is of a different wavelength of light; wherein the spectrum of light from said plurality of lasers extends from ultra-violet (UV) to near infra-red (NIR);
a spectrometer for collecting photoluminescence spectrum from said diamond responsive to inhomogeneities upon light from said lasers being directed towards said diamond;
a processor module for comparing photoluminescence spectrum collected by the spectrometer with pre-existing photoluminescence spectrum of known diamond type; and
an output module for providing an output signal indicative of the diamond type of said diamond, upon a predetermined threshold of correlation between the photoluminescence spectrum from said diamond and said pre-existing photoluminescence spectrum responsive to inhomogeneities of known diamond type.

2. A system according to claim 1, wherein the spectrometer collects photoluminescence spectrum intensity data from said diamond whilst all lasers are simultaneously activated.

3. A system according to claim 1 or claim 2, wherein said inhomogeneities include colour centers, inclusion, defects, crystallinity inconsistency, deformation of crystal lattice, internal stress, internal stress, impurities and trace elements.

4. A system according to any one of claims 1 to 3, wherein the types of diamond are natural diamonds chemical vapor deposition (CVD) synthetic diamonds, high pressure high temperature (HPHT) synthetic diamonds, and treated natural diamonds.

5. A system according to any one of the preceding claims, wherein the system comprises 3 lasers.

6. A system according to any one of claims 1 to 5, wherein the system comprises 4 lasers.

7. A system according to claim 6, wherein the lasers have wavelengths of 360 nm, 457 nm, 514 nm and 633 nm.

8. A process for determining the type of a diamond, said process including the steps of:
(i) collecting photoluminescence spectrum from said diamond responsive to inhomogeneities upon light from a plurality of lasers, wherein each laser is of a different wavelength of light; wherein the spectrum of light from said plurality of lasers extends from ultra-violet (UV) to near infra-red (NIR);
(ii) in a processor module, comparing the photoluminescence spectrum collected by the spectrometer with pre-existing photoluminescence spectrum of known diamond type; and
(iii) from an output module, responsive to predetermined threshold of correlation between the photoluminescence spectrum from said diamond and said pre-existing photoluminescence spectrum of known diamond type from step (ii) an output signal is provided indicative of the type of the diamond.

9. A process according to claim 8, wherein the spectrometer collects photoluminescence spectrum intensity data from said diamond whilst all lasers are simultaneously activated.

10. A process according to claim 8 or claim 9, wherein said inhomogeneities include colour centers, inclusion, defects, crystallinity inconsistency, deformation of crystal lattice, internal stress, internal stress, impurities, trace elements and isotopes.

11. A process according to any one of claims 8 to 10 wherein the types of diamond are natural diamonds chemical vapor deposition (CVD) synthetic diamonds, high pressure high temperature (HPHT) synthetic diamonds, and treated natural diamonds.

12. A process according any one of claims 8 to 11, wherein the pre-existing photoluminescence spectrum intensity data of known diamond type has been acquired using the system of any one of claims 1 to 6.

13. A system for automatically optimizing a collection spectrum in diamond detection, said system comprising a belt worktable, wherein a microscope objective lens and an optical device are respectively arranged on both sides of the belt worktable, a laser source is arranged in front of the microscope objective lens, a CCD sensor is arranged behind the optical device, the CCD sensor performs pre-acquisition, and the CCD sensor adjusts its own parameters according to a pre-acquisition result.

14. A system for automatically optimizing a collection spectrum in diamond detection according to claim 13, wherein in that the microscope objective lens is fixed to a support frame.

15. A system for automatically optimizing a collection spectrum in diamond detection according to claim 13 or claim 14, wherein in that the laser source is fixed to a mounting table.

16. A system for automatically optimizing a collection spectrum in diamond detection according to any one of claims 13 to 15, wherein in that the laser source comprises different lasers.

17. A system for automatically optimizing a collection spectrum in diamond detection according to any one of claims 13 to 16, wherein the CCD sensor is connected to a spectrometer.

18. A system for automatically optimizing a collection spectrum in diamond detection according to any one of claims 14 to 17, wherein the CCD sensor adopts an area array CCD.

19. A system for automatically optimizing a collection spectrum in diamond detection according to any one of claims 13 to 18, wherein the optical device is a notch filter and a fluorescence filter.

20. A diamond detection device for simultaneous co-point excitation with multiple laser lights comprising a bottom plate and a worktable, wherein the top of the bottom plate and the bottom of the worktable are both oppositely fixed with connecting rods, the connecting rods are connected by springs, the top of the bottom plate and the bottom of the worktable are both fixed with seats between the connecting rods, an air bag cooperating with the seats is arranged between the seats, a damping ring is arranged between the connecting rod and the seat, the top of the bottom plate and the bottom of the worktable are both oppositely fixed with support plates, second spring rods are oppositely fixedly connected between the support plates, elastic balls are arranged between the second spring rods, the end of the support plate is fixed with an elastic plate, a belt worktable is arranged above the worktable, a microscope objective lens and an optical device are oppositely arranged on both sides of the belt worktable, a dichroic mirror is arranged in front of the microscope objective lens, a fluorescence filter is arranged behind the optical device, the dichroic mirror, the microscope objective lens, the belt worktable, the optical device and the fluorescence filter are all fixed to the worktable through supporting rods, the supporting rod located at the bottom of the dichroic mirror is fixed to one end of a connecting rod, the other end of the connecting rod is fixed with a mounting plate, and the mounting plate is fixed with a laser source.

21. A diamond detection device for simultaneous co-point excitation with multiple laser lights according to claim 20, wherein the seats are symmetrically arranged in a vertical direction.

22. A diamond detection device for simultaneous co-point excitation with multiple laser lights according to claim 20 or claim 21, wherein the support plates are symmetrically arranged in a vertical direction.

23. A diamond detection device for simultaneous co-point excitation with multiple laser lights according to any one of claims 20 to 22, wherein the cross section of the support plate is “L” shaped.

24. A diamond detection device for simultaneous co-point excitation with multiple laser lights according to any one of claims 20 to 23, wherein the second spring rods are symmetrically arranged in a transverse direction.

25. A diamond detection device for simultaneous co-point excitation with multiple laser lights according to any one of claims 20 to 24, wherein the connecting rod is “L” shaped.

26. A diamond detection device for simultaneous co-point excitation with multiple laser lights according to any one of claims 20 to 25, wherein the laser source comprises different lasers.

27. A diamond detection device for simultaneous co-point excitation with multiple laser lights according to any one of claims 20 to 26, wherein the dichroic mirror reflects a laser wavelength, and the dichroic mirror transmits a fluorescence wavelength.

28. A diamond detection device for simultaneous co-point excitation with multiple laser lights according to any one of claims 20 to 27, wherein the optical device is a laser line filter and a spatial filter.

29. A multi-purpose optical detection system with fiber coupling comprising a bottom plate and a worktable, wherein the bottom plate and the worktable are fixedly connected by first spring rods, the side surface of the first spring rod is oppositely connected with transverse spring rods, springs are arranged both between the transverse spring rod and the bottom plate and between the transverse spring rod and the worktable, a damping cylinder is arranged outside the transverse spring rod and the springs, mounting plates and damping pads are sequentially fixed at intervals outside the damping cylinder, the top of the bottom plate and the bottom of the worktable are oppositely fixed with support plates, second spring rods are oppositely fixedly connected between the support plates, elastic balls are arranged between the second spring rods, the end of the support plate is fixed with an elastic plate, a laser source, a first fiber coupler, a first optical device, a placement table, a second optical device and a second fiber coupler are sequentially fixed from left to right on the worktable, the laser source is connected with the first fiber coupler through fibers, the second fiber coupler is connected with different spectrometers through fibers, side plates are oppositely fixed on the placement table, and the side plates are connected with fixed blocks through the springs.

30. A multi-purpose optical detection system with fiber coupling according to claim 29, wherein the first spring rods are symmetrically arranged in a transverse direction.

31. A multi-purpose optical detection system with fiber coupling according to claim 29 or claim 30, wherein the support plates are symmetrically arranged in a vertical direction.

32. A multi-purpose optical detection system with fiber coupling according to any one of claims 29 to 31, wherein the cross section of the support plate is “L” shaped.

33. A multi-purpose optical detection system with fiber coupling according to any one of claims 29 to 33, wherein the second spring rods are symmetrically arranged in a transverse direction.

34. A multi-purpose optical detection system with fiber coupling according to any one of claims 29 to 33, wherein the laser source comprises different lasers.

35. A multi-purpose optical detection system with fiber coupling according to any one of claims 29 to 34, wherein the first optical device is a lens, a dichroic mirror and a microscope objective lens, and the second optical device is a notch filter and a fluorescence filter.
